(19) United States
(12) Patent Application Publication
(10) Pub. No.: US 2020/0170932 A1
(43) Pub. Date: Jun. 4, 2020

(54) EXTENDED RELEASE L-TRI-IODOTHYRONINE SAFELY NORMALIZES KEY ELEMENTS OF MOLECULAR PATHOLOGY IN ALZHEIMERS DISEASE

(71) Applicant: JOSEPH MICHAEL ABRAMOWITZ, SAN DIEGO, CA (US)

(72) Inventor: JOSEPH MICHAEL ABRAMOWITZ, SAN DIEGO, CA (US)

(21) Appl. No.: 16/702,067

(22) Filed: Dec. 3, 2019

Related U.S. Application Data
(60) Provisional application No. 62/775,156, filed on Dec. 4, 2018.

(51) Int. Cl. A61K 9/00; A61P 25/28; A61K 38/22

(52) U.S. Cl. CPC A61K 9/0002; A61K 38/22; A61P 25/28

(57) ABSTRACT

The precise trigger mechanisms for the initiation of Alzheimer's Disease (AD) remain unidentified. However, disturbances to the balance of thyroid hormone begin in the pre-clinical stage of Alzheimer's disease. Key elements of molecular pathology in AD can be correlated with a paucity of thyroid hormone activity in the brain. A method for reversing and/or slowing progression of AD and a method for formulation of a therapeutic agent for AD are presented herein wherein an active form of thyroid hormone, T3, is formulated into an extended release dose and administered to a patient safely normalizing key elements of molecular pathology of Alzheimer's Disease.

### RELATED APPLICATIONS

This application claims the benefit of priority of U.S. Provisional Application Ser. No. 62/775,156, filed Dec. 4, 2018, of common inventorship and which is hereby incorporated by reference in its entirety.

### FIELD OF THE INVENTION

The present invention relates to Alzheimer's Disease treatment and more particularly to methods for preventing, reversing, or halting the progression of Alzheimer's Disease (AD).

### BACKGROUND

Alzheimer's disease (AD) is the most common type of dementia. The mechanisms of AD are not well understood and drug therapy focuses on restoring normal functions of neurons and glial cells. The trigger for the initiation of the long natural history of the condition we know as Alzheimer's Disease (AD) remains unconfirmed. As the disease progresses many independent brain functions become impaired. While certain pathogenetic phenomena may be due to the primary AD pathology, other pathogenetic phenomena are due to the cascading effects.

Astrocytes, oligodendrocytes, and microglia are glial cells which are vital supporting cells in the brain, surrounding neurons and providing support for and insulation between them. In the early stages of AD astrocytes have been shown to atrophy causing disruption in synaptic connections, imbalance of neurotransmitter homeostasis, neuronal death through increased excitotoxicity and in later stages they become activated and contribute to neuroinflammatory components of neurodegeneration (Verkhrtasky (1)). A key function of the astrocyte is the activation of thyroid hormone (TH). This so-called activation reaction occurs, catalyzed by the enzyme iodothyronine deiodinase type 2 (D2), via the mechanism of outer ring deiodination whereby, in a space and time dependent fashion, the polyiodinated phenoxyphenyl, L-thyroxine (tetra-iodothyronine, T4) is deiodinated at the 5′ position to form L-tri-iodothyronine, the potent and active form of thyroid hormone. The inactivation reaction occurs, catalyzed by the enzyme iodothyronine deiodinase type 3 (D3), with deiodination of the polyiodinated phenoxyphenyl at the 5 position. In AD the gene for D3 is upregulated, increasing the rate of the inactivation reaction. The activation reaction producing T3, occurs exclusively in the astrocyte. Astrocyte damage in AD impairs this reaction. Even a small impairment of this reaction can have devastating consequences for the following reasons: (i): The background, or default state of the balance of thyroid hormone is not optimum; rather maximum activation occurs only where and when it is needed; (ii): genomic and nongenomic functions of TH in the brain generally require that TH receptors have a high percentage of occupancy, believed to be at or in excess of 95% (Gereben (21)), for the effects of TH to be executed. In order for this high bar to be met, plasma levels of T3 and T4 must be within normal range and the local brain mechanisms for controlling the balance of thyroid hormone (BoTH) must be functioning normally. Astrocyte degeneration in AD causes brain levels of T3 to be inadequate, due to impairment of the activation reaction. In the neuron, TH inactivation is ramped up due to upregulation of D3 as described. In humans, TSH levels range between 0.4 and 4.4 milliunits per liter. The normal ranges of TSH, total T4, total T3, free T4, free T3 exist in a dynamic flux at this point in history with different normal ranges in different countries and even in different laboratories in the same country. It is believed that the chief reason for this is that clinical chemists and endocrinologists have only recently begun to grapple with the Gaussian issues referenced below. Also, alarmingly and distressingly, there is disagreement between the clinical chemists and the endocrinologists as to some of the specifics. For example, many clinical chemists would like to see the upper limit of TSH set around 2.5-3.0 mU/mL. The endocrinologists are reluctant to lower it below about 3.8 mU/mL. The approximate normal range for free T4 is 0.8-1.8 ng/dL. The approximate normal range of free T3 is 2.3-4.2 pg/mL. Total T4 and total T3 will not be referenced here, as they are being phased out as routine thyroid function tests. In parts of Europe, total T3 and total T4 are no longer routinely performed.

Rodent data exists suggesting a correlation between a scarcity of TH and cognitive impairment as well as AD pathology. Hypothyroidism induced in rats leads to neuropathologic signs similar to that of AD and spatial memory impairments. In adult rats, induced hypothyroidism has been found to induce amyloidogenic processing of amyloid precursor protein (APP) in the hippocampus. In a rat model of AD, administration of T3 improved histology, memory and electrophysiological activity in the cholinergic system that degenerates in AD (Sarkar {2}). In a mouse model of AD, TH was found to prevent cognitive deficit. Also in mice, Apolipoprotein-E (Apo-E) directed therapeutics have been found to rapidly clear beta-amyloid peptide(A-B) (Hu (3)).

Data also exists in human studies of AD patients suggesting the existence of an imbalance of TH in the AD brain. Increased levels of r-T3 have been found in the cerebrospinal fluid of AD patients. Reverse T3 is the chief inactivation product of T4 but not of T3. This suggests that T4 is being inactivated prior to being activated to T3 (Karimi (8)). In a study of post-mortem brains of AD patients, T3 levels were lowest in the prefrontal cortices of those decedents with the most severe neuropathology. Cohorts of AD patients have been found to have abnormally low blood levels of T3 and T4 (Decourt (4)).

Two classes of drugs have been FDA approved to treat AD. One class, cholinesterase inhibitors, include donepezil (Aricept), rivastigmine (Exelon), and galantamine (Razadyne) and are inhibitors of the enzyme acetylcholinesterase. Cholinesterase inhibitors work by lowering the brain's normal breakdown of acetylcholine, important in transformation of thought and experience into retrievable memories. The second class of AD drugs enhances the brain's sensitivity to excitatory amino acid neurotransmitter, glutamate and includes memantine (Namenda). Memantine may be combined with a cholinesterase inhibitor. These FDA approved drugs for AD may delay the progression of AD during the early and intermediate stages of the disease. Adverse effects are significant. Many clinicians and patients families are skeptical as to whether these drugs have any significant benefit.

Humans with a history of diagnosed thyroid disease are believed to have an approximately two-fold increase in the risk of Alzheimer's disease. A majority of these patients are taking thyroid hormone replacement and it is estimated that over 90% are taking T4 monotherapy. The prevalence of undiagnosed thyroid disease in the human population is significant and it increases with age. This reality makes it difficult to establish an appropriate normal range for a given thyroid laboratory blood test. When establishing a normal reference range, using classical Gaussian distribution principles, results from a cohort of normals are used. However, in the case of the above-referenced reality, a significant number will have an undiagnosed disorder of thyroid hormone production, transportation, or action in target tissue. This problem relates chiefly to T3 and T4 levels but also to TSH levels. It must be acknowledged that, with the existing normal ranges for T3 and T4, the lower half of the normal ranges contain many hidden abnormals. This reality should be known to those skilled in the arts of epidemiology and clinical chemistry.

One of the more common forms of thyroid disease leading to hypothyroidism is auto-immune thyroiditis, also known as Hashimoto's disease. The condition is presently diagnosed by the detection of significant blood levels of antibodies to the thyroid peroxidase enzyme and/or to thyroglobulin. The condition is believed to be inherited on the mothers' side through mitochondrial DNA. Some patients with autoimmune thyroiditis will also have antibodies raised against antigens in the pituitary gland, a condition known as autoimmune hypophysitis. When even partial pituitary thyrotroph failure coexists with even partial thyroid gland failure the patient is gravely hypothyroid yet usually with normal TSH, T3 and T4 levels. The reason for this is that the TSH blood test (unless measured as part of a TRH stimulation test) is not designed for the diagnosis of central hypothyroidism (hypothyroidism caused by failure of the central apparatus consisting of hypothalamus and the pituitary gland). There is also an association between autoimmune thyroiditis and Celiac disease, with its associated gluten sensitivity. It is recommended that gluten be avoided in patients with autoimmune thyroiditis, as gluten is believed to raise anti-thyroid antibody levels in these patients.

The past few decades have heralded much research and understanding of the iodothyronine deiodinase enzymes whose job it is, in space and time, to defend the optimum Balance of Thyroid Hormone (BoTH). The balance of thyroid hormone is defined as a space and time dependent phenomenon whereby a precise degree of TH activation or inactivation is called for (see FIG. 1) and achieved.

TSH, T3 and T4 (examples of the so-called thyroid function tests) are tests reflecting thyroid hormone kinetics. The term kinetics refers to what the body does to thyroid hormone, regulating its' manufacture and transport. These tests tell us nothing about the adequacy of the executive functions of thyroid hormone in the target tissues. The adequacy of the TH executive functions lies in the domain of thyroid hormone dynamics. The term dynamics refers to what thyroid hormone does to the body. There are no blood tests in clinical usage which can confirm adequacy of these executive functions. This is why the basal body temperature test, in use for decades, is still recommended. An awareness of the phenomenon of TH dynamics leads to the realization that anomalies may exist which impact what TH does to the body without being reflected in routinely ordered thyroid blood tests.

It is generally accepted that, at physiologic TH levels it is plasma T4 and not plasma T3 that is the chief negative feedback inhibitor of the hypothalamus and pituitary gland. Further, not all patients taking low to intermediate doses of exogenous T3 will experience central suppression of TSH from the T3 monotherapy. In the case of low to intermediate dose T3 monotherapy some patients manifest T3 suppression of T4 production by the thyroid gland but not suppression of the central apparatus. This suppression of thyroid gland T4 production results in a lowering of plasma T4 and a consequent rise in the TSH. This rise in TSH suggests that the patient is hypothyroid when in fact the patient is euthyroid due to the T3 monotherapy. This is confusing for clinicians, most of whom are unable to appreciate this mechanism and its benign implications.

Controversy exists over which thyroid replacement therapy is best. Is it T4 monotherapy or is it the T4/T3 combination exemplified by desiccated thyroid? The current generation of endocrinologists both in the United States and in Europe, have voiced concerns regarding T4/T3 combination therapy: (i) They believe correctly that immediate release T3 administration results in absorption spikes in plasma levels of T3 which are supra-physiologic and which put the patient at risk for cardiac arrhythmias; (ii): They point out that batches of animal-sourced desiccated thyroid have inconsistent ratios of T4 to T3; (iii): They allege that the ratio of T4 to T3 found in animal-sourced desiccated thyroid is not the same as the ratio found in humans. Consequently, these expert committees recommend that T4 monotherapy is the standard of care for TH replacement therapy. In regard to T4, it has been suggested that treatment of hypothyroidism with T4 monotherpay over many years is an independent risk factor for AD (Harper (22)). There is weak epidemiologic evidence for this. Further, T4 therapy is known to lead to ubiquitination of D2 which, while reversible, compromises the rate of the activation reaction. Aside from this research regarding ubiquitination, the broader issue of whether or not T4 monotherapy remains efficacious or possibly deleterious, after years of therapy has not been studied.

Regarding administration of T4 to patients with normal TFT's, there exists a standard of care in patients with normal TFT's who are considered at risk for thyroid cancer. These patients are treated with T4 in order to suppress TSH, the effect of said TSH on the thyroid gland being considered carcinogenic in this cohort. There exists no current standard of care for patients with normal TFT's to be treated with T3 long term.

In the brain, the hypothalamus produces thyrotropin releasing hormone (TRH) stimulating the pituitary to release thyroid stimulating hormone (TSH). The thyroid releases Thyroxine (T4) which is converted to 3,5,3′-triiodothyronine (T3), the active form of thyroid hormone (TH) by iodothyronine deiodinase type-2 (D2). The healthy astrocyte and the healthy neuron are coordinated via control mechanisms to provide the precise and optimum balance of thyroid hormone for a given microanatomic locus and time frame. The astrocyte contains iodothyronine deiodinase type-2 (D2) which converts L-thyroxine (T4) to 3,5,3′-triiodothyronine (T3) by outer ring deiodination, thus achieving TH activation. The neuron contains iodothyronine deiodinase type-3 (D-3) which converts T4 to reverse T3 (r-T3) by inner ring deiodination, thus achieving TH inactivation. Thus, in a healthy person, the coordination of activation and inactivation appropriately accommodates the required balance of thyroid hormone when maximum activation is called for and the balance is adjusted for maximum activation. Maximum activation in the healthy patient is defined here as that which provides sufficient T3 at the nuclear receptors such that 95% or more of the nuclear receptors are occupied by T3.

The half-life of D2, the chief executor of thyroid hormone activation, at around 40 min, is substantially shorter than that of D3, the chief executor of thyroid hormone inactivation, at around 12 hours. To quote Gereben (21), “D2 is considered the critical homeostatic T3 generating deiodinase due to its' substantial physiological plasticity. A number of transcriptional and posttranscriptional mechanisms have evolved to ensure limited expression and tight control of D2 protein levels, which is critical for its' homeostatic function. D2 activity/mRNA ratios are variable, indicating that there is significant posttranslational regulation of D2 expression. In fact, the decisive biochemical property that characterizes the homeostatic behavior of D2 is its' short half-life (apprx. 40 min), which can be further reduced by exposure to physiological concentrations of its' substrate, T4, and in experimental situations, rT3 or even higher concentrations of T3. This downregulation of D2 activity by substrate is a rapid and potent regulatory feedback loop that efficiently controls T3 production and intracellular T3 concentration based on the availability of T4.”

Teleologically the human organism has evolved with a strict mandate to protect the organism from unwanted thyroid hormone activation. This is evidenced by the manner in which thyroid hormone is handled in the human embryo as well as in post-natal target tissues. As the pace of the increase in human life expectancy has outstripped the capacity for evolution to adjust, this teleologic favoring of thyroid hormone inactivation over activation has become a liability in the aging human. Further, as AD patients are past their reproductive years, it is unlikely that natural selection pressures could ever correct this issue. Clearly what is needed is a treatment option comprising a therapeutic composition and method of administration that normalizes the molecular pathology of AD, said interventions preventing AD, reversing AD, or halting progression of AD, depending on the stage of AD at which intervention is begun.

### SUMMARY OF THE INVENTION

The inventor proposes that the most likely candidate for a cause of AD, as will be explained herein, acting either alone or in conjunction with other factors, is an aberration in the Balance of Thyroid Hormone (BoTH) in the human brain. T3 levels and the on demand generation of T3 are not only critical to AD. They are critical to a panoply of other syndromes, with a hitherto unrecognized association with thyroid hormone activation, known as ‘thyroid hormone dysregulation syndromes’, which are listed in U.S. Provisional Application Ser. No. 62/929,864, filed Nov. 2, 2019, and which are included here by reference.

The lower limit of normal for free T3 in patients aged 13-19 is 3.0 pg/mL (Quest diagnostics, Current Standard of Reporting). In patients over age 90, the free T3 is generally less than 2.0 (personal observations, unpublished data). In a small cohort of these patients it has been found to be clustered between 1.6 and 1.9

Thus, although no cause/effect relationship has to date been proven, it is proposed that the prime candidate for an endogenous metabolic cause of AD is an aberration in the Balance of Thyroid Hormone (BoTH) in the human brain produced by:

(i): A degradation in the Km, or reaction rate, of D2; the enzyme which catalyzes the deiodination of T4 to T3, as shown in FIG. 1, said degradation occurring progressively with increasing age and which, in certain patients, may be exacerbated by other factors, such as long term T4 monotherapy.

(ii): Abolition of the normally robust D2 activity in the astrocyte, due to astrocyte damage sustained early in AD. This astrocyte damage may occur simultaneously with the AD triggering process, or it may occur secondary to the effects of said triggering process.

(iii): Presence of abnormal amounts of TGF-B in the AD brain, a phenomenon which upregulates D3 and the inactivation reaction in the neuron, thereby degrading T4 to reverse T3, said T4 being diverted away from D2, where it is desperately needed for T3 generation. Under normal healthy circumstances, D2 in the astrocyte would reflexly upregulate. However, given that D2 is compromised as noted in (i) and (ii) above, this does not occur.

(iv): Excess r-T3 produced further compromises the BoTH in 2 ways.

(1): Elevated levels of r-T3, resulting from (iii) (Sampaolo; previously referenced) further hamstring the already compromised D2 by reducing its' half-life.

(2): Elevated levels of r-T3, resulting from (iii) (Sampaolo; previously referenced) act as a competitive blocker (a non-agonist, because its agonist activity is too weak) of T3 at the nuclear receptor.

As such, a method of preventing and/or reversing and/or halting the Alzheimers disease process is provided. In addition, a method for creating a therapeutic agent to combat AD is presented. The methods comprise formulation and administration of extended or controlled release active ingredient, wherein the active ingredient may be T3, being L-triidothyronine, liothyronine, liothyronine sodium, or similar formulations or compounds, which may safely normalize key elements of molecular pathology in Alzheimer's Disease patients. T3 or L-triidothyronine and/or similar compounds may be suitable for administration in extended release, also termed controlled release, dose formulations. In another embodiment low-dose frequent administration via oral, injectable, or other suitable route of administration to a human patient may be preferred. Whether in controlled release or low-dose treatment, formulations may be presented in vehicles not limited to a tablet, capsule, gelcap, a powder dispensed in a beverage, orally disintegrating tablet, a vial, ampule, or other container of liquid such as a solution or suspension, a lozenge, lollipop, gum, inhalers, aerosols, injectables, creams, gels, lotions, ointments, balms, eye drops, suppositories, and patches. Alternately an internal device may be implanted in the patient and release T3 over time. Active ingredient concentration or dose amount may be described as appropriate depending on weight and/or size of the patient. For instance, the dosage may be at least 1 micrograms (μg) of active ingredient, or at least 3 μg, or at least 5 μg, or at least 8 μg, or at least 10 μg, or at least 12.5 μg, or at least 25 μg, or at least 30 μg, or higher.

Extended release formulations and/or controlled or delayed-release dosage forms have been used since the 1960s to enhance performance and increase patient compliance while also potentially minimizing unwanted side effects. The dosage forms may comprise those configured to release the active ingredient over a four-hour period, or over an eight-hour period, or a twelve, twenty-four hour, thirty-six hour, or even forty-eight hour period. Alternately the release may be a delayed release in that the active ingredient doesn't reach significant levels in the blood until about one to four hours after dosing with release over the next twenty-four to twenty-six hours or more including up to thirty-six or forty-eight hours.

Total twenty-four hour or daily intake of the active ingredient may be at least 1 μg, or 2 μg of active ingredient, or at least 3 μg, or at least 5 μg, or at least 8 μg, or at least 10 μg, or at least 12.5 μg, or at least 15 μg, or at least 18 μg, or at least 25 μg, or at least 30 μg, per day or higher. In other embodiments, the unit dosage form may comprise one or more extended-release dosage forms which are configured to release the active ingredient over a period of days such as in the case of the internal device as described above.

Oral extended release or controlled release formulations may be of several types. Matrix type extended release systems or diffusion-controlling membranes, or other extended release technologies may be employed. Non-active inert ingredients, which may also be excipients for drug delivery and/or needed for formulation may be included. In another embodiment, T3 may be formulated together with T4, or levothyroxine or L-thyroxine, to maintain normal TSH levels. Levothyroxine is a synthetic thyroid hormone that may be available under the names Levothroid, Levovyxl, Levo-T, Synthroid, Tirosint, and Unithroid. Thus, it is appreciated that the optimum pharmaceutical in the instant case may be an extended release formulation of a T3 and T4 combination with variable T4/T3 ratios allowing for customized patient formulation. The T4/T3 ratio may be as much as 40:1, or 40:3, or 40:6, or 40:9, or 40:12, or 40:15, or 60:15, or other ratios.

In other embodiments a method wherein the concentration of L-tri-iodothyronine agonist at the thyroid hormone receptors in the brain are increased to normal pre-AD levels is described. Further, a method further comprising maintaining a patient on a lowest therapeutic concentration, wherein the lowest therapeutic concentration is defined as the dose wherein after gradual increase of T3 concentration to the patient, symptoms of cognitive impairment either lessen or disappear is described.

The method may result in blood levels of T3 more closely approaching steady state blood levels compared with the administration of an immediate release formulation of T3. Further, the method may be used in patients with TSH levels which are within the normal ranges. The method may also be used in patients with TSH levels which are outside the normal ranges.

In other embodiments, ingredients such as gluten may be omitted from the formulation. Further, a release profile beginning around 4 hours post ingestion or injection with slow release lasting to at least 20 if not 24, 36 or 48 hours is preferred. The release profile should result in the lowest possible blood levels of T3.

The described method may, in the pre-clinical patients, prevent the cognitive impairment of AD and in clinical patients the cognitive impairment of AD may be reversed or halted via the method. In AD cases more advanced than the early clinical phase, the pharmaceutical may slow progression of elements of the disease. The key elements of molecular pathology referenced above, described hereinafter, include (i) amyloid precursor protein (APP) transcriptional dysregulation; (ii) lipid and lipoprotein dysregulation: (iii) microtubular dysregulation and (iv) adrenergic dysregulation. The first two lead to excessive production, reduced brain export of beta-amyloid (A-B). The third leads to the tauopathy of neurofibrillary tangles seen in AD. Adrenergic dysregulation produces various symptoms and signs in AD. The use of an extended release preparation containing T3 avoids the adverse effects of the immediate release preparation chiefly, but not limited to, cardiac arrhythmias.

It is one objective of the present invention to provide a method for the management of Alzheimer's disease. The method provided comprises the administration of at least a T3 formulation to a human containing extended release T3, being L-triidothyronine, liothyronine, liothyronine sodium, or similar compounds, or a combination of T3 and L-thyroxine (T4) for the purposes of restoring normal thyroid hormone levels and normalizing key elements of molecular pathology associated with AD. AD may result from aberration of BoTH in the Alzheimer's patient's brain such that the method may have the advantages of: (a) preventing impending cognitive impairment due to pre-clinical Alzheimer's disease; (b) reversing cognitive impairment due to early clinical Alzheimer's disease; (c) slowing or halting the rate of cognitive decline in patients with more advanced Alzheimer's disease than (b); and/or (d) avoiding the adverse effects of immediate release L-triidothyronine. It is another objective to describe a method for formulation of a therapeutic agent used to prevent and/or treat Alzheimer's Disease. Further it is an objective of the disclosure to provide a method of production of a therapeutic agent for treatment of AD.

These and other objects, features and advantages of the present invention will become more apparent in light of the following detailed description of preferred embodiments thereof, as illustrated in the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 depicts the brain balance of thyroid hormone in a healthy patient.

FIG. 2 depicts the brain balance of thyroid hormone in Alzheimer's disease.

FIG. 3A illustrates amyloid precursor protein gene regulation in the normal brain.

FIG. 3B illustrates amyloid precursor protein gene dysregulation in the Alzheimer brain.

FIG. 4A illustrates lipid regulation in the normal brain.

FIG. 4B illustrates lipid dysregulation in the Alzheimer brain.

FIG. 5A illustrates lipoprotein regulation in the normal brain.

FIG. 5B illustrates lipoprotein dysregulation in the Alzheimer brain.

FIG. 6A illustrates microtubular metabolism in the normal brain.

FIG. 6B illustrates microtubular consequences in the Alzheimer brain.

FIG. 7A depicts endoplasmic reticulum stress and oxidative stress in the Alzheimer brain without T3 supplementation.

FIG. 7B depicts endoplasmic reticulum stress and oxidative stress in the Alzheimer brain with T3 supplementation.

FIG. 8 presents a Venn Diagram showing Alzheimer's disease and hypothyroid dementia as overlap syndromes and shows a list of causes for each condition.

FIG. 9 is a graph showing transcriptional effects of ERT3 dosing every 48 hours.

Before explaining the disclosed embodiments of the present invention in detail, it is to be understood that the invention is not limited in its application to the details of the particular arrangement shown, since the invention is capable of other embodiments. Also, the terminology used herein is for the purpose of description and not of limitation.

### DETAILED DESCRIPTION OF THE INVENTION

The therapeutic composition and method described herein is a treatment composition and method for preventing and/or reversing and/or halting progression of Alzheimer's Disease (AD) by introduction of T3 to a human patient via an extended release formulation. Alternately, a low-dose of T3 may be introduced over time. Further a combination of T3 and T4 may be administered. In addition, a method for creation of a therapeutic agent for treating AD is presented.

A scarcity of thyroid hormone (TH), being 3,5,3′-triiodothyronine or T3, is proposed here as a cause of the Alzheimer Dementia Phenotype (ADP). This deficiency may be a primary or a secondary phenomenon. As a primary phenomenon it is, jointly or severally, the primary trigger for the pathogenesis of the phenotypical Alzheimer dementia. As a secondary phenomenon it may occur regardless of the primary cause.

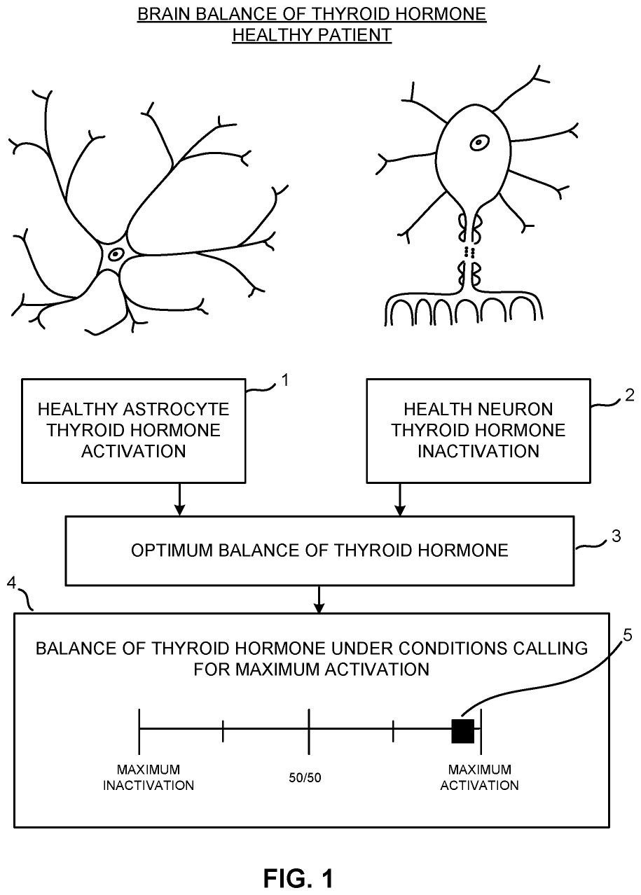

FIG. 1 depicts the brain balance of steady state of TH in a healthy normal patient. In the healthy brain, the hypothalamus produces thyrotropin releasing hormone (TRH) stimulating the pituitary gland to release thyroid stimulating hormone (TSH). The thyroid gland produces T4 and T3, releasing the hormones into the central circulation via tributaries of the superior vena cava. The ratio of T4 to T3 produced by the thyroid gland ranges from 4:1 to 9:1. Eighty to 85% of the T3 in the peripheral circulation is derived, not from thyroid gland production but, from the peripheral conversion of T4 to T3 by D2. The healthy astrocyte 1 and the healthy neuron 2 are coordinated via control mechanisms to provide the precise and optimum BoTH for a given microanatomic locus and time frame. The astrocyte 1 contains iodothyronine deiodinase type-2 (D2) which converts L-thyroxine (T4) to 3,5,3′-triiodothyronine (T3) by outer ring deiodination, thus achieving TH activation. The neuron 2 contains iodothyronine deiodinase type-3 (D-3) which converts T4 to reverse T3 (r-T3) by inner ring deiodination, thus achieving TH inactivation. Thus, in a healthy person, the coordination of activation and inactivation appropriately accommodates the required optimum balance 3 of thyroid hormone (BoTH). Under circumstances requiring maximum activation of thyroid hormone, the balance is then adjusted for maximum activation 4. Maximum activation in the healthy patient is arbitrarily defined here as that which provides sufficient T3 at the nuclear receptors such that 95% or more of the nuclear receptors 5 are occupied by T3.

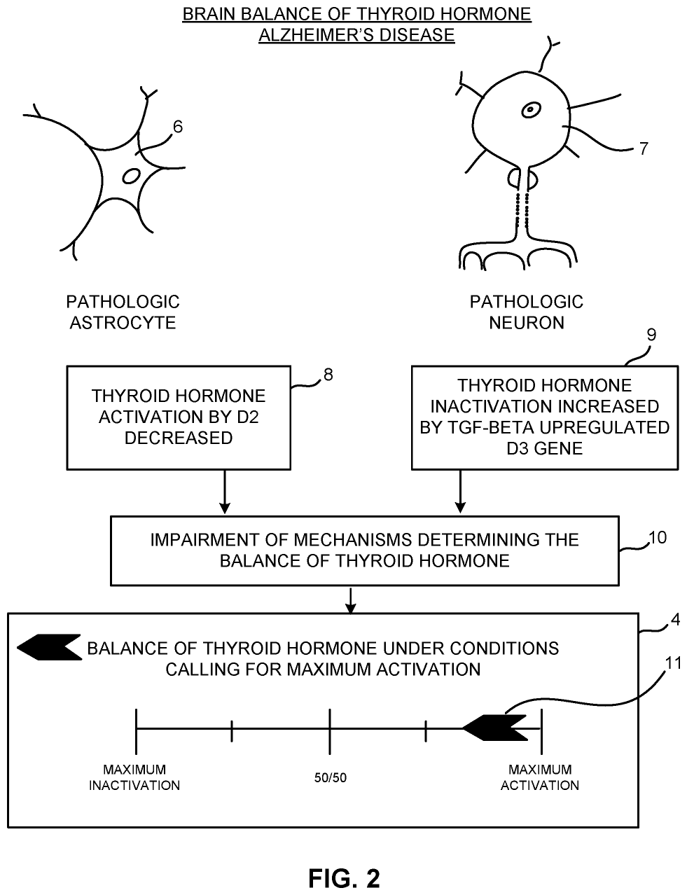

FIG. 2 depicts the brain of an Alzheimer's Disease (AD) patient. In the brain, the hypothalamus produces thyrotropin releasing hormone (TRH) stimulating the pituitary gland to release thyroid stimulating hormone (TSH). The thyroid gland releases Thyroxine (T4) which is converted to 3,5,3′-triiodothyronine (T3), the active form of thyroid hormone (TH) by iodothyronine deiodinase type-2 (D2). The healthy astrocyte and the healthy neuron are coordinated via control mechanisms to provide the precise and optimum BoTH for a given microanatomic locus and time frame. The astrocyte contains iodothyronine deiodinase type-2 (D2) which converts Thyroxine (T4) to 3,5,3′-triiodothyronine (T3) by outer ring deiodination, thus achieving TH activation. The neuron contains iodothyronine deiodinase type-3 (D-3) which converts T4 to reverse T3 (r-T3) by inner ring deiodination, thus achieving TH inactivation. Once Alzheimer's disease progresses to a critical stage, a pathologic astrocyte 6 is no longer able to prosecute the activation reaction 8 utilizing D2 with the required sufficiency. Also, at or prior to this stage the gene for D3 is upregulated in the pathologic neuron 7 increasing the inactivation reaction 9. This upregulation is accomplished by TGF-beta whose effect is increased in AD. With this decrease in TH activation and this increase in TH inactivation, there is serious impairment 10 of the mechanisms for the maintenance of the balance of thyroid hormones (BoTH). As a consequence of the foregoing, the brain is unable to generate a sufficiency of thyroid hormone activation when conditions call for maximum activation 4. As a result, the occupancy of the nuclear thyroid hormone receptors falls below the required 95% 11. This paucity of T3 at the nuclear receptor for TH in AD is the milestone which defines the onset of clinically evident cognitive impairment. As the disease progresses further, the cognitive impairment increases in inverse proportion to the to the percentage occupancy of the nuclear receptors by the progressively decreasing concentrations of T3 in the nucleus. Cognitive impairment is likely profound at a level of receptor occupancy between 85-90%.

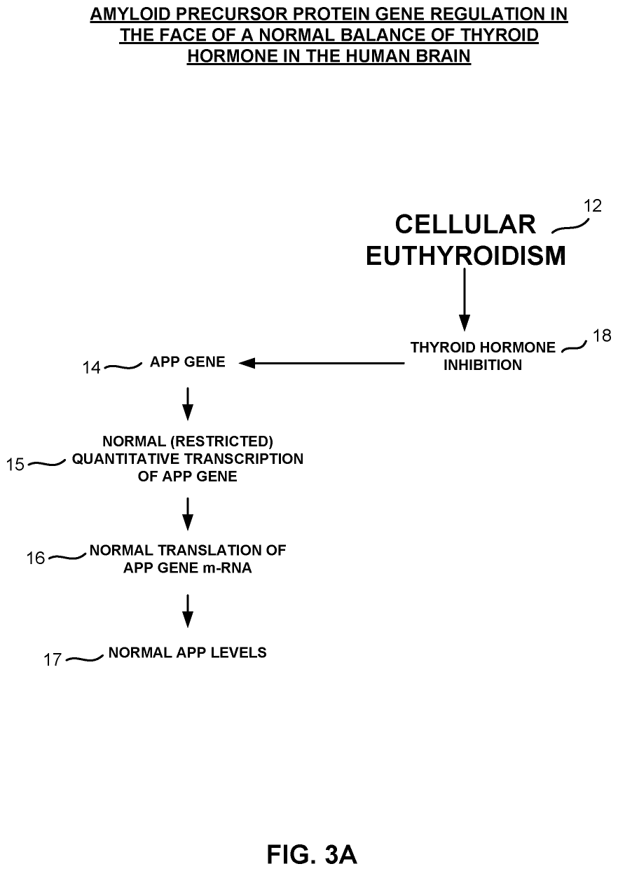

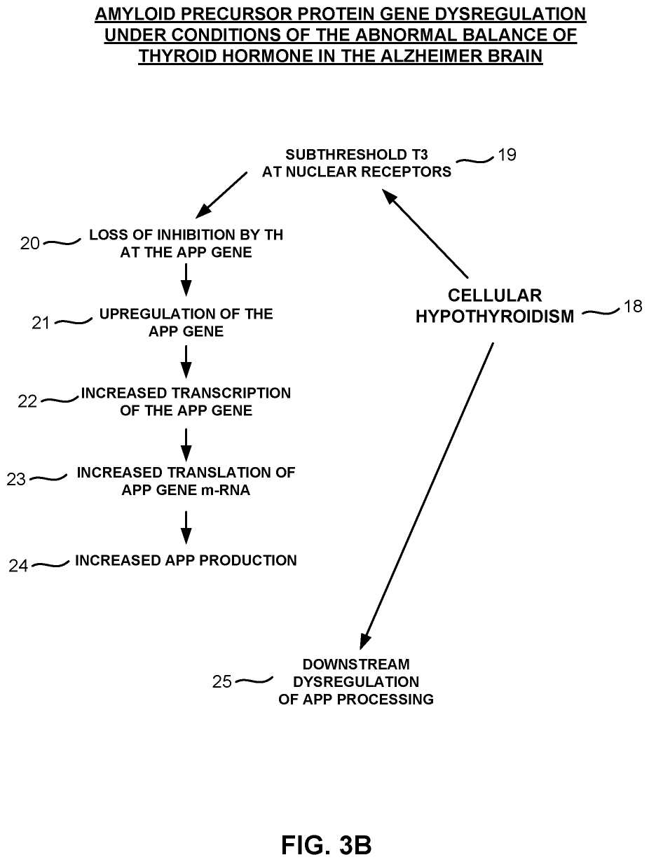

FIG. 3A depicts amyloid precursor protein gene regulation in the face of a normal brain balance of thyroid hormone, namely brain cellular euthyroidism 12. Under these normal conditions of brain cellular euthyroidism 12 thyroid hormone inhibits 13 transcription of the APP gene 14. The result is a restricted quantitative transcription of the APP gene 15. Transcripted APP gene m-RNA is also normal resulting in normal translation of the APP gene m-RNA 16. This results in normal levels of amyloid precursor protein 17.

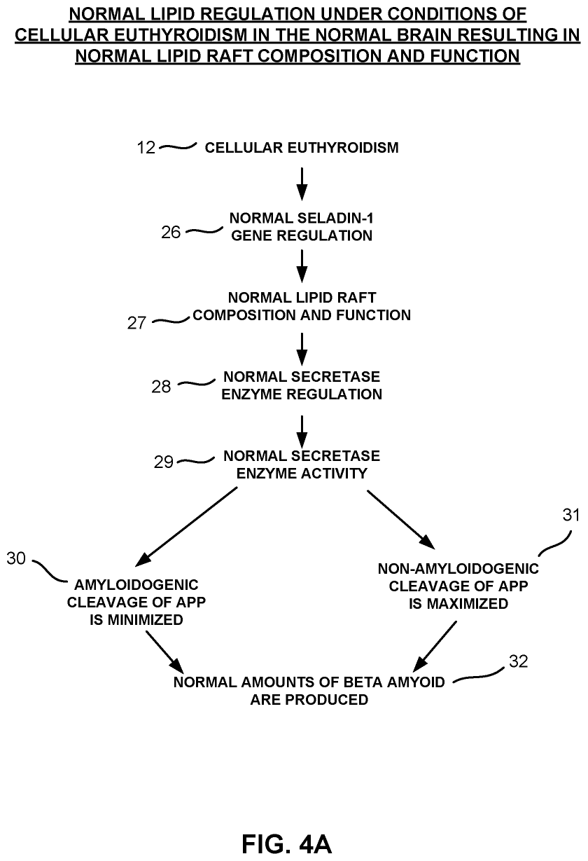

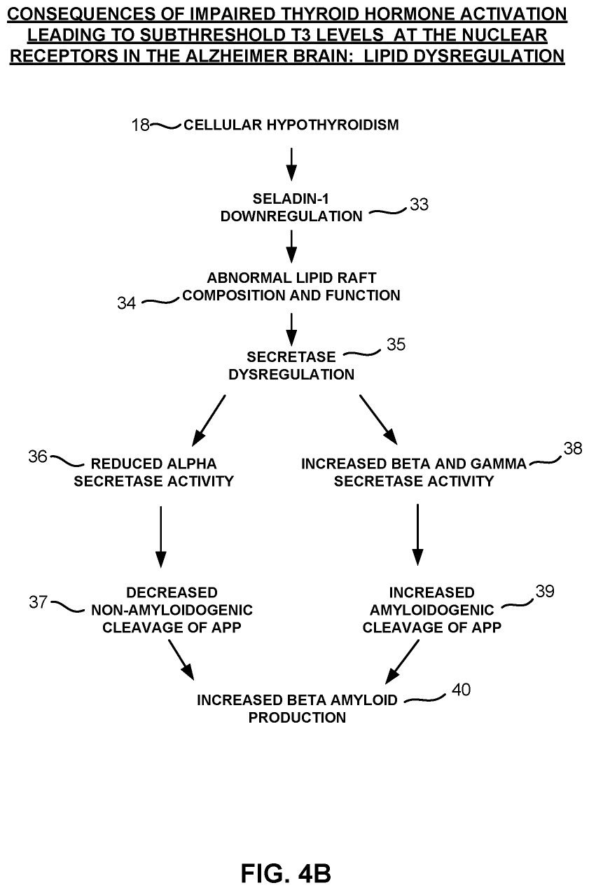

FIG. 3B depicts APP gene dysregulation in the Alzheimer brain under conditions of the abnormal balance of thyroid hormone, as referenced in FIG. 2. The cellular hypothyroidism 18 that exists results in a sub-threshold concentration of T3 at the nuclear receptors 19. This results in a loss of inhibition by TH at the APP gene 20. The APP gene upregulates 21. The transcription of APP is increased 22. Translation of APP m-RNA to assemble amyloid precursor protein is increased 23. This results in increased amounts of amyloid precursor protein being produced 24. The cellular hypothyroidism 18 also results in downstream aberrations in APP processing 25 as described in FIG. 4B.

FIG. 4A depicts normal lipid regulation under conditions of cellular euthyroidism in the normal brain resulting in normal lipid raft structure and function. The prevailing cellular euthyroidism 12 results in normal Seladin-1 gene regulation 26. This leads to the assembly of lipid rafts which have normal composition and function 27. The regulation of the secretase enzymes (alpha, beta and gamma) is normal 28. The secretase enzymes function with normal activity 29. Amyloidogenic cleavage of APP is minimized 30 while non-amyloidogenic cleavage of APP is maximized 31. Consequently normal amounts of beta amyloid are produced 32.

FIG. 4B depicts the consequences of impaired TH activation in the Alzheimer brain leading to disordered lipid metabolism causing aberrations in the normal physiology outlined in FIG. 4A. TH is a critical modulator of lipid metabolism. Lipid rafts are sub-cellular domains found in the plasma membrane, golgi and lysosomes. The composition of lipid rafts consists of a specific ratio of its constituents including, but not limited to, cholesterol and sphingolipids. Normal lipid raft functions include cleavage of APP (amyloidogenic or non-amyloidogenic) minimizing amounts of beta amyloid produced. The selective Alzheimer disease indicator gene (Seladin-1) protein is believed to be responsible for normal lipid raft composition and assembly. The gene for this protein has been found to be downregulated in regions of the human brain most affected by AD pathology (Ishida (12)). Also considered critical for Seladin-1 gene expression are the TH-beta receptor (TR-B), liver X receptor (LXR-a), insulin-like growth factor-1 (IGF-1), estrogens and androgens. In AD lipid raft composition and function are both abnormal. This is believed to be due to lower Seladin-1 gene expression as a result of the cellular hypothyroidism in the AD brain resulting from the abnormal BoTH. In the presence of cellular hypothyroidism 18 resulting from sub-threshold levels of T3 at the nuclear receptors, the Seladin-1 gene is downregulated 33. This results in abnormal lipid raft composition and function 34. This leads to dysregulation of the activity of the secretase enzymes 35. Alpha secretase activity is reduced 36 resulting in a decrease in non-amyloidogenic cleavage of APP 37. Beta and gamma secretase activity is increased 38 resulting in increased amyloidogenic cleavage of APP 39. The net result is an increase in the production of beta amyloid 40.

Normal lipoprotein function is considered essential for the breakdown of beta amyloid in the brain and for export of beta amyloid out of the brain. In the case apolipoprotein-E (Apo-E), regardless of subtype, normal TH homeostasis is required for normal Apo-E executive functions to occur. Dyslipidemia causally related to TH is not only found in AD. There is precedent for this phenomenon in the disorder of intermediate density lipoprotein (IDL), known as Fredrickson type 3 hyperlipoproteinemia. In this condition patients who are homozygous for Apo-E2 develop this form of hyperlipoproteinemia when they become hypothyroid, producing excessive amounts of intermediate density lipoprotein (IDL). TH has a shepherding relationship with the lipoproteins, regulating their production and assisting with the discharge of their duties. Thus activated TH/T3 levels are critical. The primary producers of lipoproteins in the brain are the astrocytes and the microglia, both of which sustain progressive damage beginning early in the course of AD. Brain lipoprotein production in AD is compromised on at least two levels, The TH catalyst is compromised because of sub-threshold TH activation. In addition the cells responsible for lipoprotein production, astrocytes and microglia, are incapable of normal function because they are damaged due to the Alzheimer pathology. Certain proteins have been identified as critical for the export of A-B across the blood brain barrier and into the bloodstream. Examples of these transporters are lipoprotein receptor protein-1 (LRP-1) and the ABC transporter proteins such as ABCB-1. Research has shown that the genes for a number of these proteins are upregulated by TH. An additional and important function of the microglia is export of opsonized A-B from the brain. Microglial damage in AD impairs this process. A consequence of these deficiencies, involving the transporter proteins and the microglia, is the impaired transport of A-B across the blood-brain barrier and out of the brain. This produces a bottleneck for A-B exiting the brain.

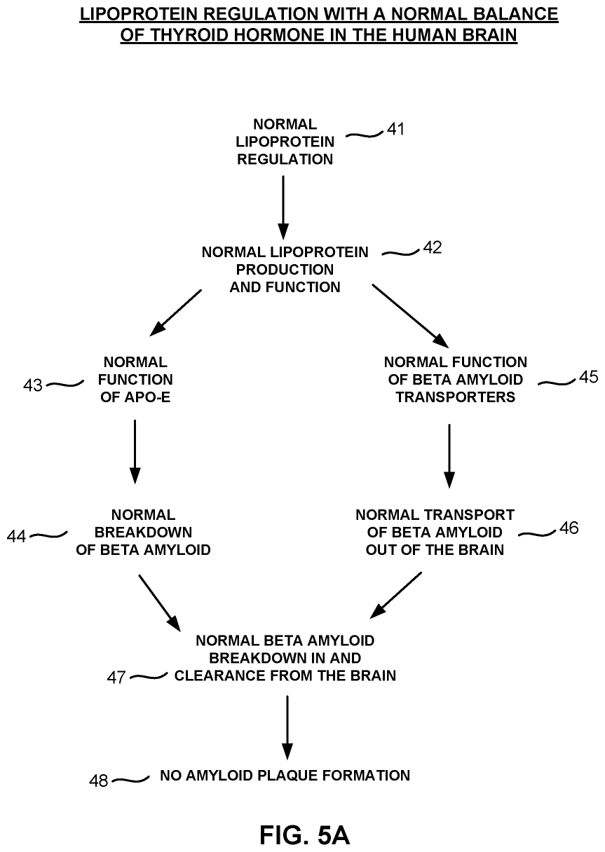

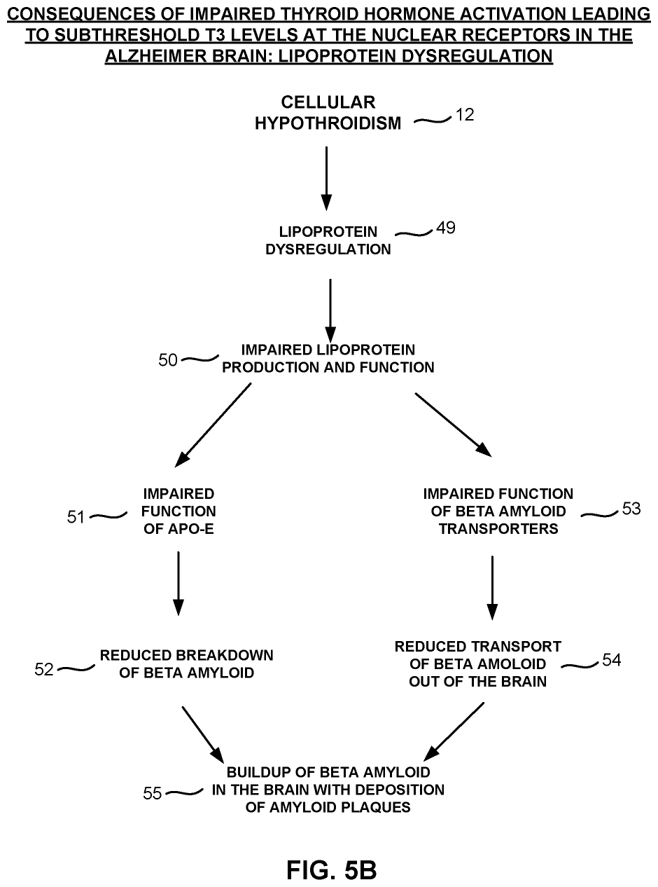

FIG. 5A depicts normal lipoprotein regulation in the human brain under conditions of brain euthyroidism. Normal lipoprotein regulation 41 results in normal lipoprotein production and function 42. This is associated with normal function of Apolipoprotein E 43 which results in normal breakdown of beta amyloid 44. Normal lipoprotein production and function is also associated with the normal function of beta amyloid transporters 45 which results in normal transport of beta amyloid out of the brain 46. There is normal brain clearance of beta amyloid 47 and consequently there is no amyloid plaque formation in the brain 48.

FIG. 5B depicts the consequences of impaired brain TH activation on the lipoprotein physiology referenced in FIG. 5A. Under conditions of brain cellular hypothyroidism, lipoprotein dysregulation 49 occurs. This results in impaired lipoprotein production and function 50. Impaired function of Apo-E 51 occurs which leads to reduced breakdown of beta amyloid 52. Further, there is impaired function of the beta amyloid transporters 53 resulting in reduced transport of beta amyloid out of the brain 54. The net effect of the foregoing is the buildup of beta amyloid in the brain with the deposition of amyloid plaques 55.

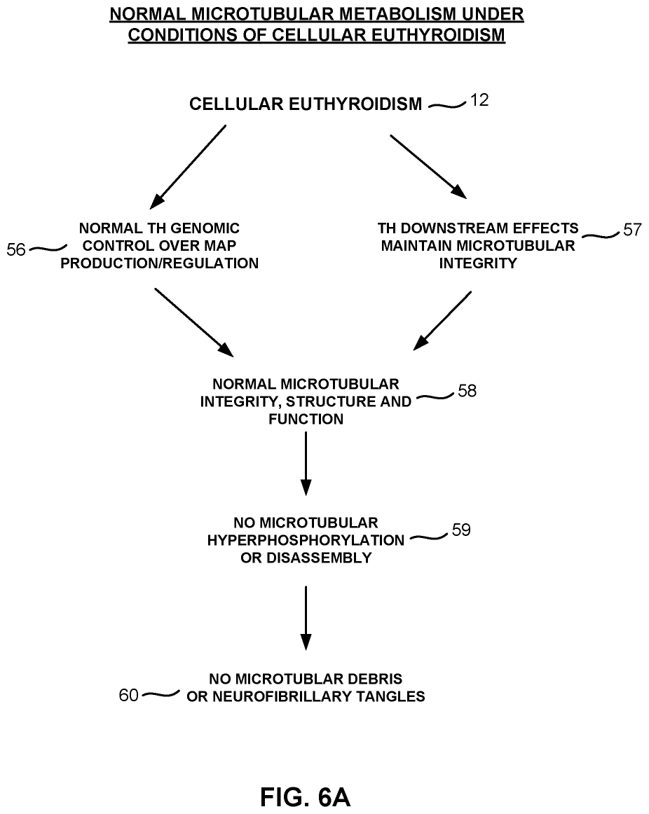

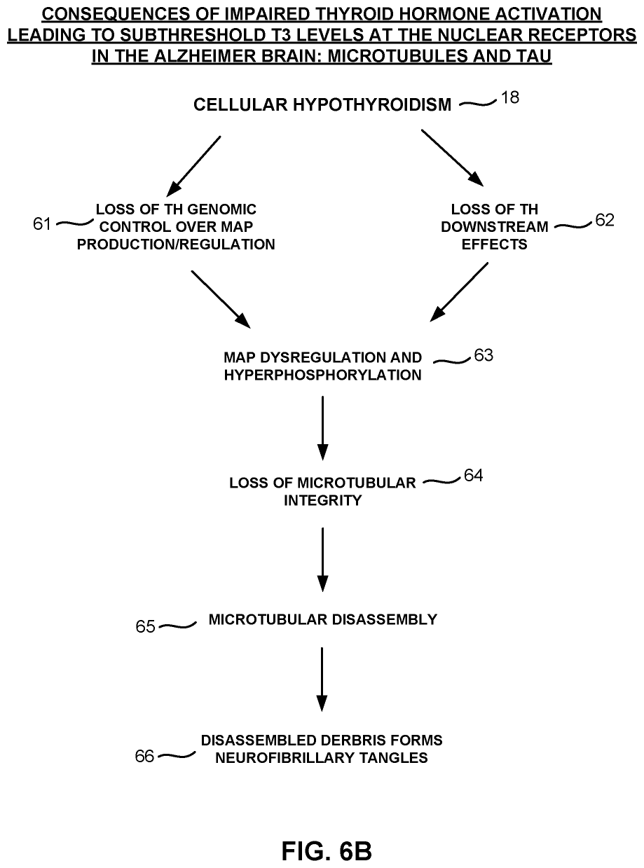

FIG. 6A depicts normal microtubular metabolism under normal conditions of brain cellular euthyroidism 12. Normal genomic regulation by TH of MAP's controls production and regulation of MAP's 56. TH downstream effects 57 on assembly and of assembled microtubules maintains microtubular integrity. Consequently normal microtubular integrity, structure and function are maintained 58. No microtubular hyperphosphorylation or disassembly occurs 59. As there is no microtubular disassembly, there is no microtubular debris and no neurofibrillary tangles 60 form.

FIG. 6B depicts the physiologic steps shown in FIG. 6A under conditions of the brain cellular hypothyroidism 18 of AD. There is loss of the normal TH genomic control over MAP's 61. The subthreshold levels of TH result in the absence of the salutary downstream effects 62 of TH on microtubular integrity. MAP dysregulation and hyperphosphorylation 63 occur. This leads to a loss of microtubular integrity 64 which progresses to microtubular disassembly 65. This leads to debris which is deposited as neurofibrillary tangles 66.

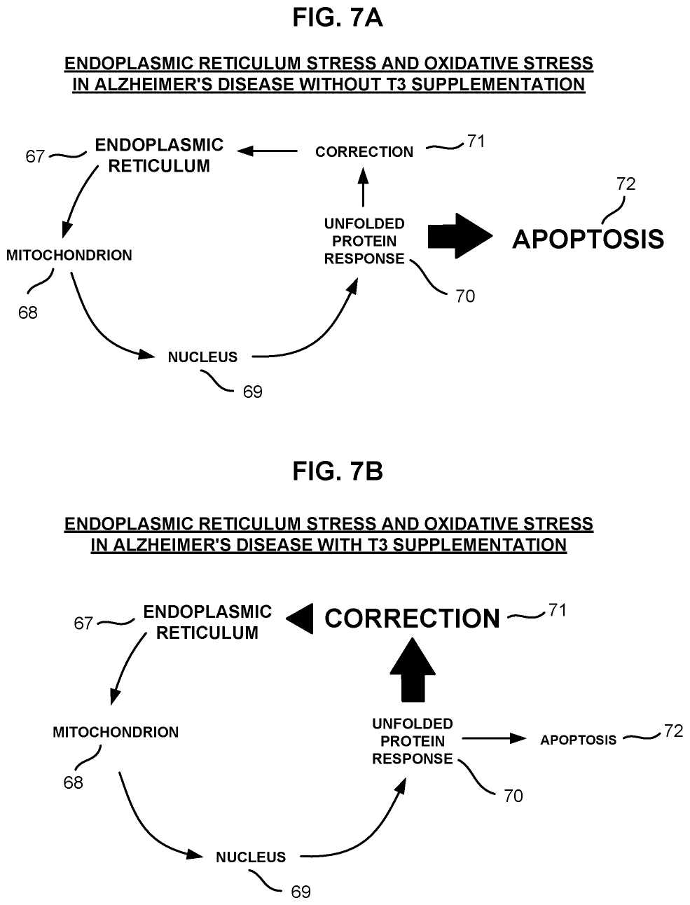

FIG. 7A shows the endoplasmic reticulum stress and oxidative stress which are found in numerous disease processes including Alzheimers disease and type 2 diabetes. The endoplasmic reticulum 67 is shown in the schematic together with the mitochondrion 68 and the nucleus 69. Endoplasmic reticulum stress is caused by cellular hypothyroidism, the Thr92Ala polymorphism of D2, as well as conditions unrelated to TH dynamics. Regardless of the cause of endoplasmic reticulum stress, the molecular biologic derangements are complex. One of the most important of these derangements is disruption of the activity of D2 which is resident in the ER. The reason that this is important is that T3 is the most potent physiologic regulator of mitochondrial function, both qualitatively and quantitatively. The disruption of D2 activity eliminates the one remedy for the coexisting oxidative stress and, at the same time, worsens the oxidative stress further. The unfolded protein response (UPR) 70 is a natural cellular stress response related to and triggered by endoplasmic reticulum stress. It is conserved in all mammalian species. The UPR aims at restoring normal function to the cell by halting protein translation, degrading misfolded proteins and activating signaling pathways involved in normal protein folding. In the event that these objectives are not achieved within a certain time frame, the UPR shifts its goal to apoptosis by promoting cell death. Under circumstances of brain cellular hypothyroidism, the UPR lacks the resources to correct the situation. Correction 71 in the ER is minimal and apoptosis 72 is the main outcome.

FIG. 7B shows endoplasmic reticulum stress and oxidative stress when T3 73 is administered to compensate for the deficient D2 activity in the ER. The schematic shows the endoplasmic reticulum 67 the mitochondrion 68 and the nucleus 69. The supplemented T3 73 is now able to fulfill its' molecular biologic mandate. The UPR 70 triggered is now able to maximize the required correction 71 in the ER and to minimize apoptosis 72. Qualitative and quantitative mitochondrial activity is restored, ameliorating the oxidative stress.

The noradrenerigic neurotransmitter system is dependent on normal thyroid hormone activity for normal function. A deficient amount of thyroid hormone activity leads to downregulation of the noradrenergic system. This leads to attenuated postsynaptic effects and a failure to prosecute the noradrenergic mandate. In AD this phenomenon accounts for various signs and symptoms. There are aberrations of the diurnal rhythm including insomnia and daytime somnolence. Depression and/or anxiety may occur. Drooping of the upper eyelid (ptosis) is frequently seen in AD. The levator palpebrae superioris muscle, the elevator of the upper eyelid, is partially innervated by the sympathetic nervous system. As a testament to the veracity of the instant invention, administering T3 to patients with AD results in a rapid, within days, and dramatic wide-eyed countenance and an appearance of increased alertness. Rarely, as an additional manifestation of adrenergic dysregulation, skin picking may occur which may be minimized by T3 administration.

Due to the deleterious effects of low T3/activated TH in AD patients, the instant application is drawn to a method for treating AD, as well as a method of creating a therapeutic agent for treatment of AD, via administration of T3 or L-Triiodothyronine, also known as Liothyronine, or Liothyronine Sodium, known by the brand/trade name Cytomel. Liothyronine (L-Triiodothryonine) and 3,5,3′-Triiodothyronine (T3/Activated TH) are nearly identical to one another, but Liothyronine is more potent and better absorbed orally. Liothyronine has been developed into a prescription medication and preparation known as Cytomel, Tiromel, Tertroxin, as well as others.

Because T3 is a stimulating hormone, excess can lead to cardiac complications which include cardiac hypertrophy, arrhythmias and high output heart failure. Even in the absence of sustained chronic T3 excess, immediate release T3, with its' supraphysiologic post-absorptive plasma levels, may produce cardiac arrhythmias, chiefly supraventricular. Therefore, immediate release T3 is not suitable, especially for older patients. Absorption of T3 (L-triidothryonine or liothyronine) is 90% with peak levels reached one to two hours following ingestion. Serum concentration, or amount of drug in circulation, may rise by 250% to 600%. T3 may have a short half-life being only nineteen hours. Single dose, immediate release T3 ingestion may place a patient at risk for cardiac arrhythmias, chiefly but not limited to supra-ventricular arrhythmias, and potentially other adverse effects. Consequently, the American Geriatric Society has designated desiccated thyroid (containing immediate release T3) as fitting the Beers Criteria, indicating a need for avoidance, or use with caution, in older adults.

A method for treating AD with T3 being L-triiodothyronine, liothyronine, liothyronine sodium, or similar formulations in an extended release system allows patients to be treated for AD in a safe manner. Extended release caplets or tablets or other suitable vehicle for administration, being via oral, injectable, or other suitable route of administration to a human patient, not limited to a tablet, capsule, gelcap, a powder dispensed in a beverage, orally disintegrating tablet, a vial, ampule, or other container of liquid such as a solution or suspension, a lozenge, lollipop, gum, inhalers, aerosols, injectables, creams, gels, lotions, ointments, balms, eye drops, suppostitories, and patches, with the minimum T3 dose, tailored to the individual patient for body weight and age for instance, being at least 2 μg, or at least 5 μg, or at least 10 μg, or at least 12.5 μg, or at least 15 μg, or 20 μg, or at least 25 μg, or at least 30 μg per day or higher, overcomes these concerns resulting in lower serum concentration levels. Alternately a drug dispensing device may be implanted either sub-dermally or otherwise and configured to release T3 in a slow manner. The post absorptive blood levels of this extended release T3 could more closely resemble a steady state or constant level of T3 in the blood rather than a high spike in post-absorptive blood levels of the immediate release formulation, thereby avoiding supra-physiologic or high serum concentration of T3 levels in the blood. This tailoring to the individual patient may be achieved by the treating physician making judgments based on the patients' symptoms and signs as well as results of thyroid function tests, as well as T3, T4, and TSH, and/or TH level monitoring.

A subset of patients taking T3 monotherapy (T3 without T4) will show thyroid function tests (TFT's) which demonstrate an apparently spurious rise in thyroid stimulating hormone (TSH). This occurs because the levels of plasma T3 generated in these patients are insufficient to result in central negative feedback inhibition/suppression of TSH. This central negative feedback inhibition/suppression of TSH is primarily a T4 mediated phenomenon, mediated by T3 only at higher blood levels in some patients. The origin of the apparently spurious rise in TSH is explained here. While the therapeutic T3 level in this subset of patients is too low for central negative feedback inhibition/suppression of TSH, it is not too low to produce negative feedback directly to the thyroid gland. This effect reduces production and secretion of T4 by the thyroid gland. As a consequence, the plasma level of T4 falls, reducing the central feedback inhibition/suppression of T4 on the central apparatus and thus the TSH rises. This phenomenon results in an elevated TSH, suggesting a hypothyroid state, when in fact the patient is euthyroid by virtue of the T3 treatment.

Therefore, in another embodiment, T3 may be formulated together with T4, or the two may be given in separate formulations at the same time, thereby maintaining T4 levels with a sufficiency such that central negative feedback inhibition is maintained and a normal TSH is preserved. Thus, it is appreciated that the optimum pharmaceutical in the instant case is an extended release formulation of a T4/T3 combination with variable T4/T3 ratios allowing for customized patient formulation. The T4/T3 ratio may be as much as 40:1, or 40:3, or 40:6, or 40:9, or 40:12, or 40:15, or 60:15, or other ratios.

Extended release formulations and/or delayed-release dosage forms have been used since the 1960s to enhance performance and increase patient compliance while also potentially minimizing unwanted side effects. The dosage forms may comprise those configured to release the active ingredient over a four-hour period, or over an eight-hour period, or a twelve, or twenty-four-hour period, or thirty-six hour period, or even forty-eight hour period. In other embodiments, the unit dosage form may comprise one or more extended-release dosage forms which are configured to release the active ingredient over a period of days. Matrix type extended release systems or diffusion-controlling membranes, or other extended release technologies may be employed. Non-active inert ingredients for drug delivery may be included in formulations.

Matrix type systems may be based on hydrophilic polymers wherein the drugs and excipients, being non-active inert ingredients, are mixed with polymer such as hydroxypropyl methylcellulose (HPMC) and hydroxypropyl cellulose (HPC) and then formed as a tablet by conventional compression. Water diffuses into the tablet, swells the polymer and dissolves the drug or active ingredient, whereupon the drug may diffuse out being released into the body. This type of controlled or extended release technology is open to mechanical stress from food substances which may lead to increased release rate and a higher risk of dose-dumping. These systems also require a large amount of excipient and drug loading is comparatively low.

Diffusion-controlling membranes is another method of obtaining extended or controlled release of active ingredients. With this technology, a core that may be pure active ingredient, or mixture of active ingredient and excipient(s), is coated with a permeable polymeric membrane. Water diffuses through the membrane and dissolves the drug which then diffuses out through the membrane at a rate determined by the porosity and thickness of the membrane. Membrane polymers may be those such as ethylcellulose.

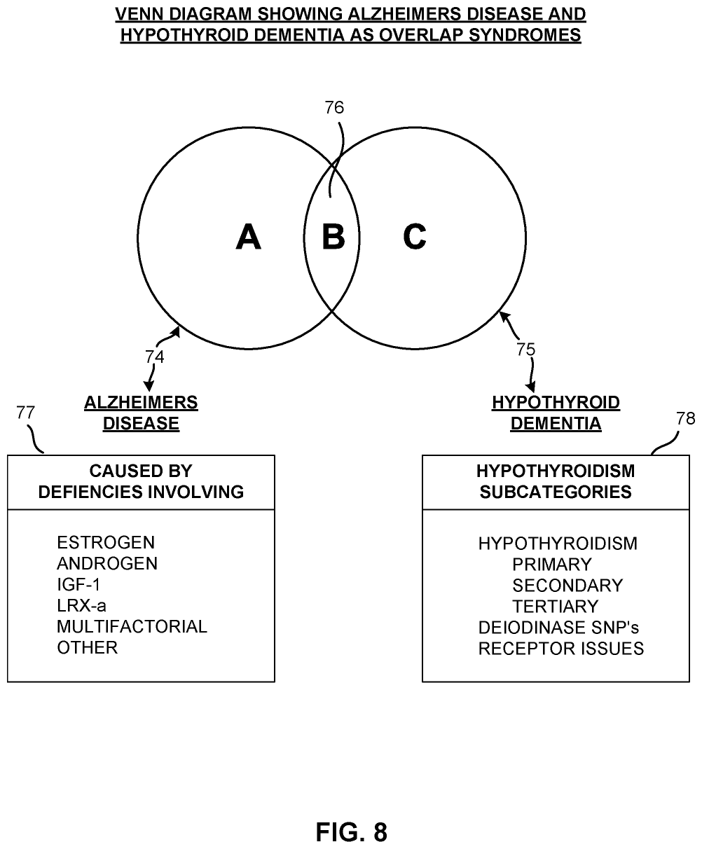

FIG. 8 is a Venn diagram presenting Alzheimer's disease 74 (AD) and hypothyroid dementia 75 (HD) as overlapping syndromes with a listing of purported etiologies in each category. Circle A 74 represents AD. This is a dementia with symptoms consistent with AD rather than other dementias. It is also a dementia in which elements of thyroid hormone kinetics and dynamics are clearly normal. Circle B 75 represents HD. This may be a dementia resembling AD but with least one key element of thyroid hormone kinetics or dynamics is clearly abnormal. The region of overlap 76 represents dementia consistent with AD but with elements of thyroid hormone kinetics and/or dynamics not clearly definitive one way or the other. Amyloid, or beta-amyloid, scans would be expected to be positive in both AD and HD. Proposed etiologic factors for AD 77 include deficiencies involving estrogen in the female, androgen in the male, liver X receptor (LXR-a) which is a nuclear receptor, insulin like growth factor-1 (IGF-1), which is a downstream growth hormone agonist, multifactorial (involving more than one of the foregoing) and potentially other factors as yet unknown. An important part of the instant invention relates to the chemical moieties mentioned here as proposed etiologic factors in AD. Estrogen is a critical factor in females with AD. The role of testosterone in the male is less clear. LXR-a IGF-1 are also believed to be important, as are other chemical moieties whose role has not yet been correlated with AD causation. These other proposed chemical moieties which are not TH act on the same elements of molecular pathology as have been described for TH in the instant invention. As such they may act as surrogates for TH actions. What this means is that a sufficiency of one or other in a patient susceptible to AD may compensate for an otherwise critical T3 brain deficit. The described method for treatment of AD, and therapeutic formulation presented herein, may also be applicable to HD, other hypothyroid conditions and other thyroid hormone dysregulation syndromes referenced above, including type 2 diabetes mellitus. Hypothyroid sub-categories 78 include primary hypothyroidism, due to thyroid gland insufficiency and including autoimmune thyroiditis, or Hashimotos disease, secondary hypothyroidism due to pituitary dysfunction, tertiary hypothyroidism due to hypothalamic dysfunction, single nucleotide polymorphisms of the iodothyronine deiodinase enzymes and TH receptor aberrations. Further, Celiac disease, with its attendant intolerance to gluten, is a comorbidity of autoimmune thyroiditis, wherein gluten consumption is believed to raise the levels of thyroid autoantibodies in these patients. Hence gluten may be excluded from the formulation of the optimum pharmaceutical for the instant invention.

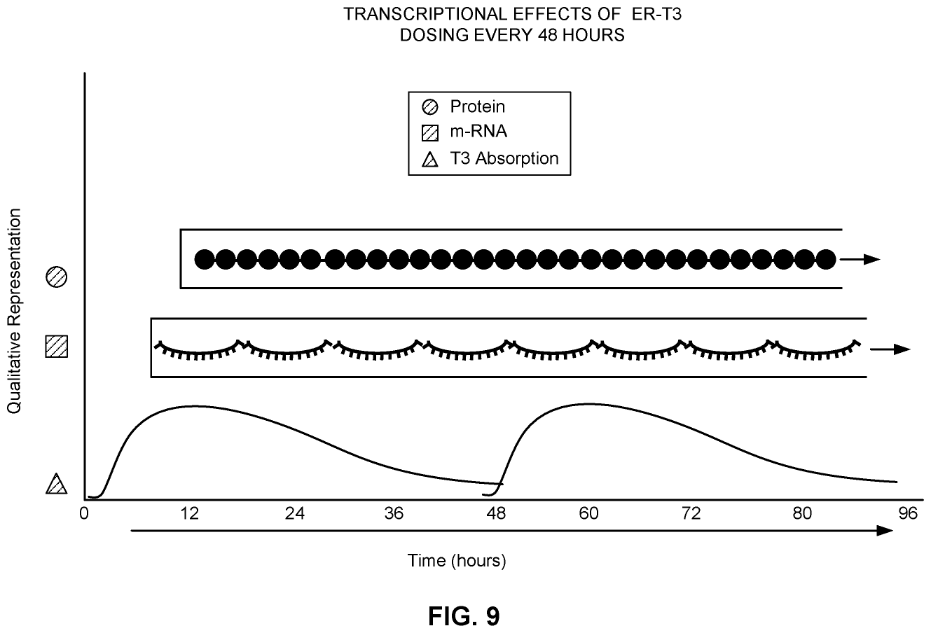

With reference to FIG. 9. it has become known that certain compounding pharmacies (University Compounding Pharmacy, San Diego Calif. 92101; personal communication) are using technology which results in release of T3 which takes 2-8 hours to completion. This is not optimal for reasons stated elsewhere. The potency of T3 is such that a more prolonged release profile is preferred. In the event that the release profile suggested below is not technically feasible, then the objectives of the instant invention will still be met completely by a dose reduction and/or a shortening of the dosing interval. The optimum ERT3 release profile for the instant invention could be:
1. A profile beginning around 2-4 hours and lasting to about 20-24 hours.
2. A profile that results in physiologic blood levels of T3 which persist for 24-36 hours following dose administration. It has been referenced that the half-life of T3 in humans is 19 hours.
3. A release profile as in (1) & (2) which results in the lowest possible plasma levels of T3.

It is now appropriate to delve into the specifics of the art located at the opposite end of the release spectrum, said art constituting a portion of the art of the present invention. This is necessary to answer the following two questions:
1. What is the maximum dosing interval for ER T3 which accomplishes the goals, genomic and non-genomic, of the present invention? This involves the pharmacokinetics of the pharmaceutical of the instant invention and relates to its plasma half-life.
2. What is the time period, of the activity of the genomic and non-genomic effects triggered by the formulation of the instant invention, during which these beneficial effects of ER T3 are active? This involves the pharmacodynamics of the pharmaceutical of the instant invention and, for purposes of clarity of discussion here, this will be referred to as the transcriptional half-life.

It is proposed here that, subject to research confirmation, the maximum dosing interval for the formulation of the instant invention for use in AD is 48 hours. Notwithstanding the fact that confirmatory research may indicate that this maximum dosing interval is longer than 72 hours, the safety margin would not be expected to be further enhanced and the efficacy would be expected to be less. This 48 hour maximum dosing interval may be contrasted with other methods as described in U.S. Provisional Application Ser. No. 62/775,156, of which is claimed priority to and described herein, of the formulation of the instant invention. Thus the preferred dosing for ERT3 for AD may be either once every 24 or once every 48 hours, although this should not exclude other options. This 48 hour maximum dosing interval should be contrasted with that for a different application of the formulation of the instant invention, that for enhanced glycemic control in Type 2 diabetes, where the preferred dosing interval of the pharmaceutical is less, possibly every 12 hours.

It should be acknowledged that AD and DM often coexist, appearing to create a conflict as to ER T3 dosing. When this occurs, the dose chosen should be at the discretion of the treating physician. It will be appreciated that numerous different controlled release embodiments may be appropriate based on the concepts embodied by the instant invention. This matter is beyond the scope here. The omission of further detail on this tangential matter here does not affect the spirit or scope of the invention. The disadvantages of immediate release T3, which does not represent the art of the present invention, have been explained.

FIG. 9 is a schematic showing transcriptional effects of ERT3 dosing every 48 hours. The lowest graphic 79 represents plasma levels of the ERT3 pharmaceutical dosed every 48 hours, the X-axis extending out to 96 hours. The middle graphic 80 represents the genomic effect of TH generating the production of m-RNA for the hypothetical protein. The upper graphic 81 represents the translational protein synthesis levels from the m-RNA. It will be noted that according to this hypothetical, the half-life of T3 of 19 hours in combination with the not insignificant transcriptional half-life of T3 depicted here, a continuous and ongoing transcription with associated m-RNA production as well as continuous and ongoing m-RNA translation with the associated protein synthesis are provided. This occurs with dosing either once every 24 hours or once every 48 hours.

Although the present invention has been described with reference to the disclosed embodiments and example, numerous modifications and variations can be made and still the result will come within the scope of the invention. No limitation with respect to the specific embodiments disclosed herein is intended or should be inferred.

### REFERENCES

1. Astrocytes in Alzheimer's Disease, VERKHRTASKY et al, Neurotherapeutics, 2010 October; 7(4): 399-412
2. SARKAR et al., Involvement of L-Triiodothyronine in Acetylcholine Metabolism in Adult Rat Cerebrocortical Synaptosomes, Hormone and Metabolic Research 33(5): 270-5, Jun. 21, 2001
3. HU, et al, BACE1 deletion in the adult mouse reverses preformed amyloid deposition and improves cognitive functions, Journal of Experimental Medicine, 14 Feb. 2018
4. DECOURT et al., BACE1 Levels by APOE Genotype in Non-Demented and Alzheimer's Post-Mortem Brains), Current Alzheimer's Research 2013 March:10 3:309-15
5. American Geriatrics Society (AGS) 2012 Beer's Criteria Update Expert Panel. AGS Updated Beer's Criteria for Potentially Inappropriate Medication Use in Older Adults. J. Am Ger Soc April (60) 4, 616-31
6. SAMPAOLO S, CAMPOS-BARROS A, MANZIOTTI G, CARLOMAGNO S, SANNINO V, AMATO G, CARELLA C, Di IORIO G (2005) Increased cerebrospinal fluid levels of 3,3′,5′ triiodothyronine in patients with Alzheimer's disease J Clin Endocrinol Metab January; 90 (1): 198-202
7. DAVIS J D, PODOLANCZUK A, DONAHUE J E Stopa E, HENNESSY J V, LUO L G, LIM Y P, STERN R A (2008) Thyroid hormone levels in the prefrontal cortex of post-mortem brains of Alzheimer's disease patients. Curr. Aging Sci December 1(3); 175-81
8. KARIMI F, HAGHIGHI A B, PETRAMFAR P (2011) Low levels of tri-iodothyronine in patients with Alzheimer's disease. Iran J Med Sci December 36 (4): 322-3
9. GUSSEKLOO J, VAN EXEL, E, DE CRAEN A J M, MEINDERS A E, FROLICH, M, WESTENDORP R G J, (2004) Thyroid Staus, Disability and Cognitive Function, and Survival in Old Age. JAMA December 292 (21): 2591-99
10. BELAKAVADI M, DELL J, Grover G J, FONDELL J D (2011) Thyroid hormone suppression of beta amyloid precursor protein gene expression in the brain involves multiple epigenetic regulatory events. Mol Cell Endocrinol June 6; 339(1-2); 72-80
11. O'BARR S A, O H J S, M A C, BRENT G A, SCHULTZ J J (2006) Thyroid hormone regulates endogenous amyloid-beta precursor protein gene expression and processing in both in vitro and in vivo models. Thyroid December; 16 (12): 1207-13
12. ISHIDA E, HASHIMOTO K, OKADA S, YAMADA M, MORI M (2013) Crosstalk between thyroid hormone receptor and liver X receptor in the regulation of selective Alzheimer's disease indicator-1 gene expression. PLoS One 2013; 8(1):e54901. doi: 10.1371/journal.pone.0054901 (Epub)
13. CORDY J M, HOOPER N M, TURNER A J (2006) The involvements of lipid rafts in Alzheimer's disease. Mol Membr Biol January-February; 23(1): 111-22
14. SON-SUN Yoon, SANGMEE Ahn Jo (2012) Mechanisms of Amyloid-B Clearance: Potential Therapeutic Targets for Alzheimer's Disease. Biomol Ther (Seoul). May; 20(3): 245-55
15. KANEKIYO T, CIRRITO J R, LIU C C, SHINOHARA M, L I J, SCHULER D R, SHINOHARA M, HOLTZMAN D M, BU G (2013) Neuronal Clearance of Amyloid-B by Endocytic Receptor LRP-1. J Neurosci December 4; 33(49): 19276-83. Doi: 10.1523/J Neurosci 3487-13.2013
16. SAGARE A P, DEANE R, ZLOKOVIC B V (2012) Low-density lipoprotein receptor-related protein 1: a physiological A-B homeostatic mechanism with multiple opportunities. Pharmacol Ther October; 136(1):94-105. Doi: 10.1016/j.pharmthera.2012.07.008. Epub 2012 Jul. 20
17. European Thyroid Expert Committee Article: FLIERS, E et al European Thyroid Association and Thyroid Federation International Joint Position Statement on the Interchangeability of Levothyroxine products in EU Countries. European Thyroid J. 2018 October; 7(5): 238-242
18. American Thyroid Expert Committee Article: JONKLAAS, J et al: Guidelines for treatment of hypothyroidism: Prepared by the American Thyroid Association task force on thyroid hormone replacement. Thyroid 2014 Dec. 1 24(12): 1670-1751
19. Laboratory Procedure Manual: Free T3, University of Washington Medical Center. Dept. of Laboratory Medicine, Author: WALSH, M. September 2006.
20. Metabolism of Thyroid Hormone. PEETERS R, VISSER T. Endotext; NCBI Bookshelf. www.endotext.org.
21. GEREBEN. Endoc. Rev. 2008 December; 29(7): 898-938. Cellular and Molecular Basis of Deiodinase-Regulated Thyroid Hormone Signaling
22. HARPER P C, ROE C M J Ger. Psych. Neurol. 2010 March: 23(1):63. Thyroid medication use and subsequent development of dementia of the Alzheimer Type.
23. NUNEZ J. 1985 Neurochem. Int. 7(6) 959-68. Microtubules and brain development: The effects of thyroid hormones.
24. JAE HOON MOON 2013 Thyroid September; 23(9) 1057-65. Decreased Expression of Hepatic Low-Density Lipoprotein-Related Protein 1 in Hypothyroidism: A Novel Mechanism of Atherogenic Dyslipidemia in Hypothyroidism.
25. VALLEJO C G 2005 Am. J. Physiol. Endocrinol. Metab. July; 289(1): E87-94 Epub 2005 Feb. 15. Thyroid hormone regulates tubulin expression in mammalian liver. Effects of deleting thyroid hormone receptor-alpha or -beta.

What is claimed is:

1) A method for preventing and reversing and halting progression of Alzheimer's Disease, the method comprising the steps of:
a) providing T3 in a controlled release formulation; and
b) administering said controlled release formulation to a human patient.

2) The method of claim 1 further comprising the step of providing T4 in the controlled release formulation.

3) The method of claim 2, wherein the ratio of T4 to T3 is not more than 40 to 1.

4) The method of claim 2, wherein the ratio of T4 to T3 is not more than 40 to 3.

5) The method of claim 2, wherein the ratio of T4 to T3 is not more than 40 to 6.

6) The method of claim 2, wherein the ratio of T4 to T3 is not more than 40 to 9.

7) The method of claim 2, wherein the ratio of T4 to T3 is not more than 40 to 12.

8) The method of claim 2, wherein the ratio of T4 to T3 is not more than 40 to 15.

9) The method of claim 2, wherein the ratio of T4 to T3 is not more than 60 to 15.

10) The method of claim 2, wherein the ratio of T4 to T3 is not more than 60 to 50.

11) The method of claim 1, wherein the formulation is free of gluten.

12) The method of claim 1, wherein administration causes concentration of L-tri-iodothyronine agonist at the thyroid hormone receptors in the brain of the patient are increased to normal levels.

13) The method of claim 1 further comprising maintaining a patient on a lowest therapeutic concentration, wherein the lowest therapeutic concentration is defined as the dose wherein after gradual increase of T3 concentration to the patient, symptoms of AD either lessen or disappear.

14) The method of claim 1, wherein administration results in blood levels of T3 more closely approaching steady state blood levels compared with the administration of an immediate release formulation of T3.

15) The method of claim 1, wherein the patient has TSH levels which are within the normal ranges.

16) The method of claim 1, wherein the patient has TSH levels which are outside the normal ranges.

17) A method for production of a therapeutic agent, the method comprising the steps of incorporating an active ingredient comprising T3 into a controlled release formulation.

18) The method of claim 14 further comprising the step of providing T4 in the therapeutic agent.

19) The method of claim 14, wherein the therapeutic agent is free of gluten.
